(19) United States
(12) Patent Application Publication
(10) Pub. No.: US 2015/0246899 A1
(43) Pub. Date: Sep. 3, 2015

(54) PROCESS FOR THE PREPARATION OF DABIGATRAN ETEXILATE OR PHARMACEUTICALLY ACCEPTABLE SALT THEREOF

(71) Applicant: RANBAXY LABORATORIES LIMITED, New Delhi, Delhi (IN)

(72) Inventors: Anandam Vempali, Nellore (IN); Sudhir Singh Sanwal, Kangra (IN); Balaguru Murugesan, Coimbatore (IN); Swargam Sathyanarayana, Gurgaon (IN); Rajesh Kumar Thaper, Jammu (IN); Mohan Prasad, Gurgaon (IN)

(73) Assignee: RANBAXY LABORATORIES LIMITED, New Delhi, Delhi (IN)

(21) Appl. No.: 14/430,303

(22) PCT Filed: Sep. 30, 2013
(86) PCT No.: PCT/IB2013/059016

§ 371 Date: Mar. 23, 2015

(30) Foreign Application Priority Data
Sep. 28, 2012 (IN) 3065/DEL/2012
Sep. 28, 2012 (IN) 3066/DEL/2012

(51) Int. Cl. C07D 401/12; C07C 53/48; C07C 53/10; C07D 213/75; C07C 309/19

(52) U.S. Cl. CPC C07D 401/12; C07D 213/75; C07C 309/19; C07C 53/10; C07C 53/48

(57) ABSTRACT

The present invention relates to a process for the preparation of dabigatran etexilate. The present invention also relates to trifluoroacetate salt of dabigatran etexilate and a process for its preparation. The present invention further relates to crystalline Form I and crystalline Form II of trifluoroacetate salt of dabigatran etexilate and processes for their preparation. The present invention further relates to a process for the preparation of pharmaceutically acceptable salts, including methanesulfonate salt, of dabigatran etexilate.

### FIELD OF THE INVENTION

The present invention relates to a process for the preparation of dabigatran etexilate. The present invention also relates to trifluoroacetate salt of dabigatran etexilate and a process for its preparation. The present invention further relates to crystalline Form I and crystalline Form II of trifluoroacetate salt of dabigatran etexilate and processes for their preparation. The present invention further relates to a process for the preparation of pharmaceutically acceptable salts, including the methanesulfonate salt, of dabigatran etexilate.

### BACKGROUND OF THE INVENTION

The drug substance used in the commercial drug product formulation of Pradaxa® is the methanesulfonate salt of dabigatran etexilate, which is chemically designated as β-Alanine, N-[[2-[[[4-[[[(hexyloxy)carbonyl]amino]iminomethyl]phenyl]amino]methyl]-1-methyl-1H-benzimidazol-5-yl]carbonyl]-N-2-pyridinyl-, ethyl ester, methanesulfonate salt of Formula I.

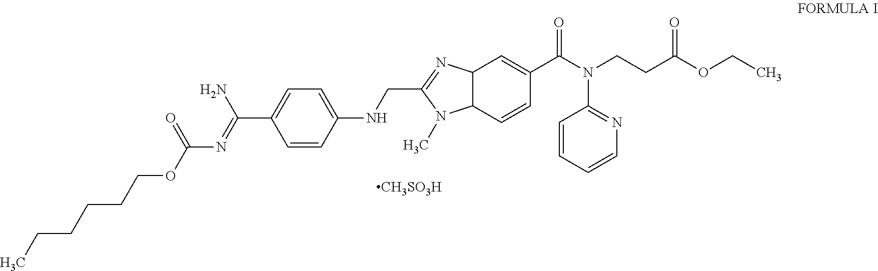

Dabigatran etexilate of Formula II

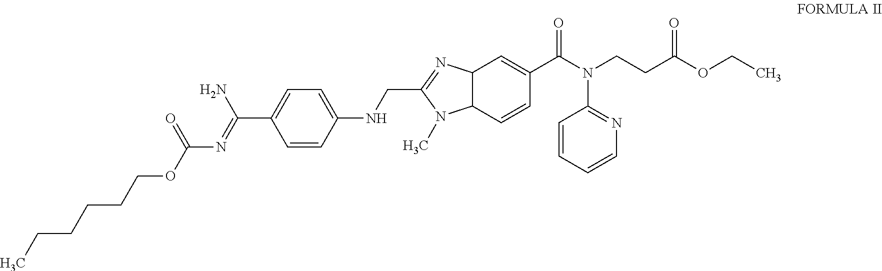 [SMILES: CCCCCCOC(=O)/N=C(\N)c1ccc(NCC2=NC3C=C(C(=O)N(CCC(=O)OCC)c4ccccn4)C=CC3N2C)cc1]

is an etexilate prodrug of dabigatran ethyl ester of Formula III

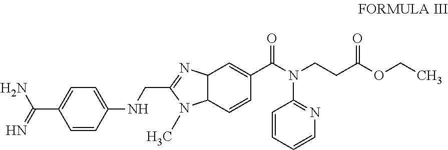 [SMILES: CCOC(=O)CCN(C(=O)C1=CC2N=C(CNc3ccc(C(=N)N)cc3)N(C)C2C=C1)c1ccccn1]

which is a prodrug of dabigatran of Formula IV

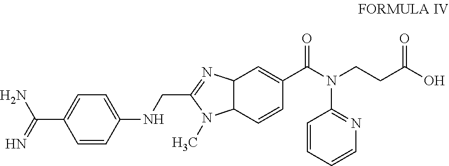 [SMILES: CN1C(CNc2ccc(C(=N)N)cc2)=NC2C=C(C(=O)N(CCC(=O)O)c3ccccn3)C=CC21]

which is a direct thrombin inhibitor. Dabigatran etexilate is indicated to reduce the risk of stroke and systemic embolism in patients with non-valvular atrial fibrillation. It may be used alone or in combination with other therapeutic agents.

Processes for the preparation of dabigatran etexilate or its different salts are described in U.S. Pat. No. 6,087,380; European Patent Publication No. EP 1 870 100 (equivalent to CA 2,476,054); and PCT Publication Nos. WO 2006/114415 (equivalent to US 2006/0247278), WO 2008/043759, WO 2012/044595, WO 2012/027543, WO 2008/059029, WO 2011/110876, WO 2011/110478, and WO 2006/131491 (equivalent to US 2006/276513).

### SUMMARY OF THE INVENTION

The present invention relates to a process for the preparation of dabigatran etexilate. The present invention also relates to trifluoroacetate salt of dabigatran etexilate and a process for its preparation. The present invention further relates to crystalline Form I and crystalline Form II of trifluoroacetate salt of dabigatran etexilate and processes for their preparation. The present invention further relates to a process for the preparation of pharmaceutically acceptable salts, including methanesulfonate salt, of dabigatran etexilate.

### BRIEF DESCRIPTION OF THE DRAWINGS

FIG. 1 depicts the X-ray powder diffraction (XRPD) pattern of the crystalline Form I of trifluoroacetate salt of dabigatran etexilate obtained according to Example 3.

FIG. 1A provides the XRPD pattern of the crystalline Form I of trifluoroacetate salt of dabigatran etexilate depicted in FIG. 1.

FIG. 2 depicts the XRPD pattern of the crystalline Form II of trifluoroacetate salt of dabigatran etexilate obtained according to Example 4.

FIG. 2A provides the XRPD pattern of the crystalline Form II of trifluoroacetate salt of dabigatran etexilate depicted in FIG. 2.

FIG. 3 depicts the XRPD pattern of the crystalline Form of methanesulfonate salt of dabigatran etexilate obtained according to Example 6.

FIG. 3A provides the XRPD pattern of the crystalline Form of methanesulfonate salt of dabigatran etexilate depicted in FIG. 3.

### DETAILED DESCRIPTION OF THE INVENTION

A first aspect of the present invention provides a process for the preparation of acetic acid salt of dabigatran ethyl ester of Formula V

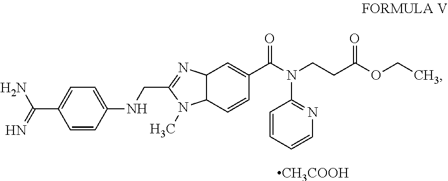

wherein the process comprises:
a) reacting the compound of Formula VI

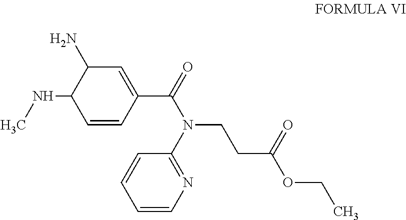 [SMILES: CCOC(=O)CCN(C(=O)C1=CC(N)C(NC)C=C1)c1ccccn1]

with the compound of Formula VII

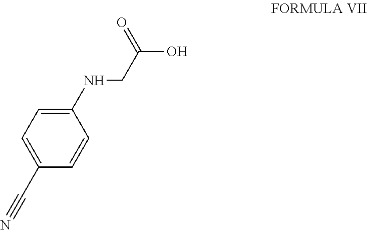 [SMILES: N#Cc1ccc(NCC(=O)O)cc1]

to prepare the compound of Formula VIII;

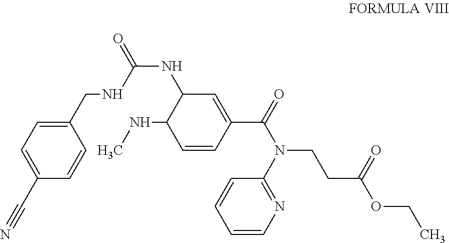 [SMILES: CCOC(=O)CCN(C(=O)C1=CC(NC(=O)NCc2ccc(C#N)cc2)C(NC)C=C1)c1ccccn1]

b) optionally isolating the compound of Formula VIII from the reaction mixture;
c) converting the compound of Formula VIII to the compound of Formula IX;

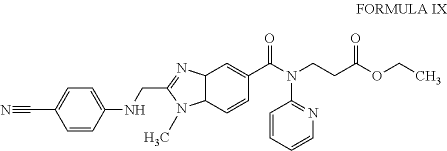 [SMILES: CCOC(=O)CCN(C(=O)C1=CC2N=C(CNc3ccc(C#N)cc3)N(C)C2C=C1)c1ccccn1]

d) converting the compound of Formula IX to the acetic acid salt of dabigatran ethyl ester of Formula V; and
e) isolating the acetic acid salt of dabigatran ethyl ester of Formula V from the reaction mixture.

The compound of Formula VI and compound of Formula VII may be reacted in the presence of a condensing agent and a solvent. The condensing agent may be selected from the group comprising carbonyl diimidazole, phosgene, triphosgene, or mixtures thereof. Preferably, the condensing agent is carbonyl diimidazole. The solvent may be selected from the group consisting of ethers, ketones, esters, or mixtures thereof. The ether solvent may be selected from the group comprising tetrahydrofuran and diisopropyl ether. The ketone solvent may be selected from the group comprising acetone, ethyl methyl ketone and ethyl butyl ketone. The ester solvent may be selected from the group comprising ethyl acetate and propyl acetate. Preferably, the solvent is tetrahydrofuran. The compound of Formula VI and compound of Formula VII may be reacted in the presence of butylated hydroxyl toluene. The compound of Formula VI and the compound of Formula VII are reacted at about 10° C. to about 80° C., for example, about 20° C. to about 60° C. The compound of Formula VI and the compound of Formula VII are reacted for about 30 minutes to about 4 hours, for example, about 1 hour to about 3 hours. The compound of Formula VIII obtained by the reaction of the compound of Formula VI and the compound of Formula VII may optionally be isolated.

The compound of Formula VIII may be converted to the compound of Formula IX in the presence of a cyclizing agent and a solvent. The cyclizing agent may be, for example, acetic acid. The acetic acid may also act as a solvent. The solvent may be selected from the group consisting of water, esters, alcohols, ketones, or mixtures thereof. The alcohol solvent may be selected from the group comprising ethanol and 2-propanol. The ketone solvent may be selected from the group comprising acetone, ethyl methyl ketone, and ethyl butyl ketone. The ester solvent may be selected from the group comprising ethyl acetate and propyl acetate. Preferably, the solvent is ethyl acetate, 2-propanol, or mixtures thereof. The conversion of the compound of Formula VIII to the compound of Formula IX is carried out at about 10° C. to about 100° C., for example, about 20° C. to about 90° C. The conversion of the compound of Formula VIII to the compound of Formula IX is carried out for about 6 hours to about 25 hours, for example, about 10 hours to about 20 hours. The compound of Formula IX may optionally be isolated by filtration, decantation, evaporation, distillation, or combinations thereof.

The compound of Formula IX may optionally be converted to the compound of Formula X.

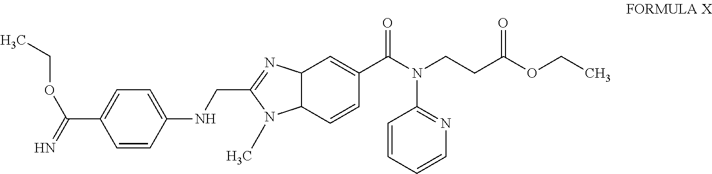 [SMILES: CCOC(=N)c1ccc(NCC2=NC3C=C(C(=O)N(CCC(=O)OCC)c4ccccn4)C=CC3N2C)cc1]

The conversion of the compound of Formula IX to the compound of Formula X is carried out in the presence of hydrochloric acid and a solvent. The solvent may be selected from the group consisting of alcohols, amides, esters, or mixtures thereof. The alcohol solvent may be selected from the group comprising ethanol and 2-propanol. The amide solvent may be dimethyl formamide. The ester solvent may be selected from the group comprising ethyl acetate and propyl acetate. Preferably, the solvent is dimethylformamide, ethanol, or mixtures thereof. The conversion of the compound of Formula IX to the compound of Formula X is carried out at about −20° C. to about 60° C., for example, about −10° C. to about 50° C. The conversion of the compound of Formula IX to the compound of Formula X is carried out in about 6 hours to about 25 hours, for example, about 10 hours to about 20 hours.

The compound of Formula IX or compound of Formula X is converted to the acetic acid salt of dabigatran ethyl ester of Formula V in the presence of ammonium acetate, and a solvent. The solvent may be selected from the group consisting of water, alcohols, ketones, esters, or mixtures thereof. The alcohol solvent may be selected from the group comprising ethanol and 2-propanol. The ketone solvent may be selected from the group comprising acetone and ethyl methyl ketone. The ester solvent may be selected from the group comprising ethyl acetate and propyl acetate. Preferably, the solvent is water, ethanol, acetone, or mixtures thereof. The conversion of the compound of Formula IX or the compound of Formula X to the acetic acid salt of dabigatran ethyl ester of Formula V is carried out at about 0° C. to about 60° C., for example, about 5° C. to about 50° C. The conversion of the compound of Formula IX or the compound of Formula X to acetic acid salt of dabigatran ethyl ester of Formula V is carried out in about 1 hour to about 25 hours, for example, about 10 hours to 20 hours.

The acetic acid salt of dabigatran ethyl ester of Formula V may exist in the form of the mono-acetate or di-acetate. The acetic acid salt of dabigatran ethyl ester of Formula V may be isolated by filtration, decantation, evaporation, distillation, or combinations thereof.

A second aspect of the present invention provides a process for the preparation of dabigatran etexilate of Formula II

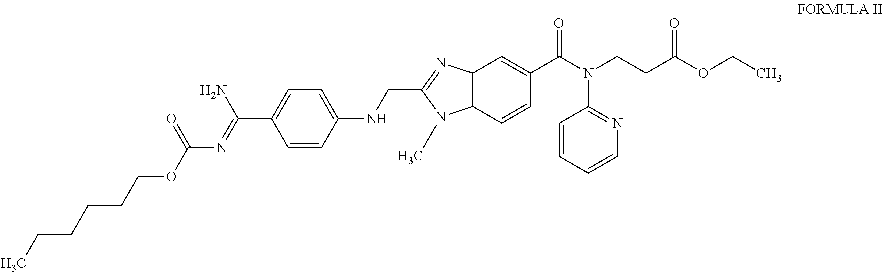 [SMILES: CCCCCCOC(=O)/N=C(\N)c1ccc(NCC2=NC3C=C(C(=O)N(CCC(=O)OCC)c4ccccn4)C=CC3N2C)cc1]

or a salt thereof wherein the process comprises:
a) reacting the compound of Formula VI

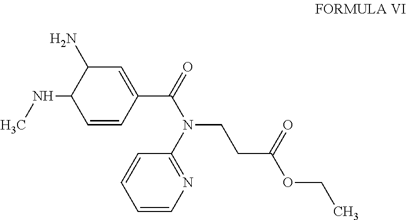 [SMILES: CCOC(=O)CCN(C(=O)C1=CC(N)C(NC)C=C1)c1ccccn1]

with the compound of Formula VII

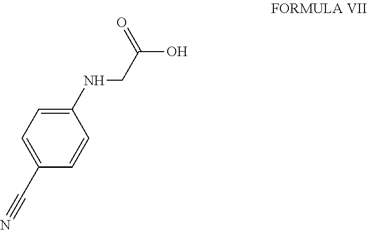 [SMILES: N#Cc1ccc(NCC(=O)O)cc1]

to prepare the compound of Formula VIII;

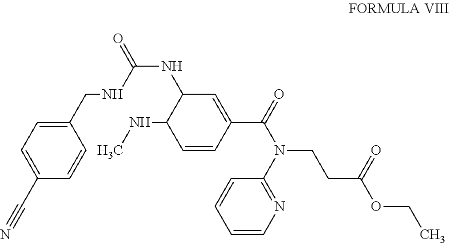 [SMILES: CCOC(=O)CCN(C(=O)C1=CC(NC(=O)NCc2ccc(C#N)cc2)C(NC)C=C1)c1ccccn1]

b) optionally isolating the compound of Formula VIII from the reaction mixture;
c) converting the compound of Formula VIII to the compound of Formula IX;

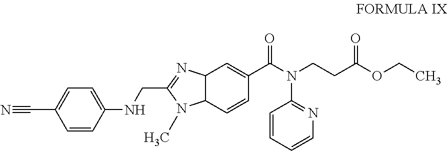 [SMILES: CCOC(=O)CCN(C(=O)C1=CC2N=C(CNc3ccc(C#N)cc3)N(C)C2C=C1)c1ccccn1]

d) converting the compound of Formula IX to the acetic acid salt of dabigatran ethyl ester of Formula V;
e) optionally isolating the acetic acid salt of dabigatran ethyl ester of Formula V from the reaction mixture;
f) reacting the acetic acid salt of dabigatran ethyl ester of Formula V with a reagent capable of providing an etexilate group; and
g) isolating the dabigatran etexilate of Formula II or a salt thereof from the reaction mixture.

The compound of Formula VI and the compound of Formula VII may be reacted as described in the first aspect. The compound of Formula VIII obtained by the reaction of the compound of Formula VI and the compound of Formula VII may optionally be isolated. The compound of Formula VIII may be converted to a compound of Formula IX as described in the first aspect. The compound of Formula IX may optionally be isolated by filtration, decantation, evaporation, distillation, or combinations thereof.

The compound of Formula IX may optionally be converted to the compound of Formula X as described in the first aspect. The compound of Formula IX or the compound of Formula X may be converted to the acetic acid salt of dabigatran ethyl ester of Formula V as described in the first aspect.

The acetic acid salt of dabigatran ethyl ester of Formula V may exist in the form of mono-acetate or di-acetate. The acetic acid salt of dabigatran ethyl ester of Formula V may be isolated by filtration, decantation, evaporation, distillation, or combinations thereof.

The acetic acid salt of dabigatran ethyl ester of Formula V is converted to dabigatran etexilate of Formula II or its salt by contacting with a reagent capable of providing an etexilate group and a solvent. Preferably, the salt of dabigatran etexilate is trifluoroacetate salt. The reagent capable of providing the etexilate group may be n-hexyl chloroformate. The solvent may be selected from the group consisting of water, ethers, halogenated hydrocarbons, esters, ketones, or mixtures thereof. The ether solvent may be selected from the group comprising tetrahydrofuran, diisopropyl ether, and methyl t-butyl ether. The halogenated hydrocarbon solvent may be dichloromethane. The ester solvent may be ethyl acetate. The ketone solvent may be selected from the group comprising acetone and ethyl methyl ketone. Preferably, the solvent is tetrahydrofuran either alone, or in combination with water. The n-hexyl chloroformate may be used either as a solid or in solution form with tetrahydrofuran.

The acetic acid salt of dabigatran ethyl ester of Formula V is contacted with n-hexyl chloroformate is the presence of an organic base or an inorganic base. The organic base may be selected from the group comprising ethylamine and diisopropyl ethyl amine. The inorganic base may be selected from the group comprising sodium carbonate and potassium carbonate. Preferably, the base is potassium carbonate.

The acetic acid salt of dabigatran ethyl ester of Formula V is contacted with the n-hexyl chloroformate at a temperature of about 10° C. to about 40° C., for example, about 15° C. to about 25° C. The acetic acid salt of dabigatran ethyl ester of Formula V is contacted with the n-hexyl chloroformate for about 3 hours to about 6 hours, for example, about 4 hour to about 6 hours.

The reaction mixture of the acetic acid salt of dabigatran ethyl ester of Formula V and the n-hexyl chloroformate may be subjected to carbon treatment. The reaction mixture of the acetic acid salt of dabigatran ethyl ester of Formula V and the n-hexyl chloroformate may optionally be treated with butylated hydroxytoluene. The solvent may be recovered from the reaction mixture and the reaction mixture used as such for the next step.

The reaction mixture obtained in the previous step may be treated with a suitable reagent to prepare the salt of dabigatran etexilate. Preferably, the salt of dabigatran etexilate is trifluoroacetate salt of dabigatran etexilate salt of Formula XI.

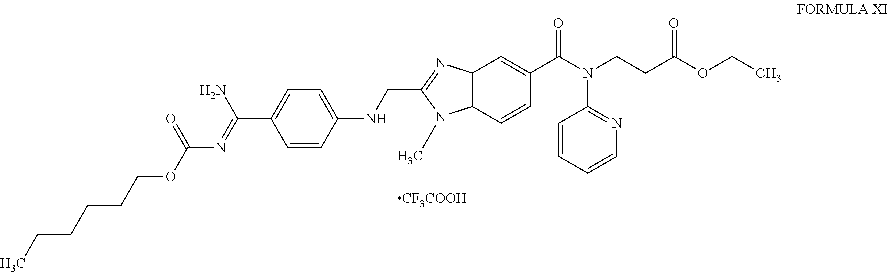 [SMILES: CCCCCCOC(=O)/N=C(\N)c1ccc(NCC2=NC3C=C(C(=O)N(CCC(=O)OCC)c4ccccn4)C=CC3N2C)cc1 ; "·CF3COOH"]

The salt of dabigatran etexilate, for example, trifluoroacetic acid salt, may be prepared in the presence of a solvent selected from the group consisting of ketones, esters, alcohols, or mixtures thereof. The ketone solvent may be selected from the group comprising acetone, methyl butyl ketone, and methyl isopropyl ketone. The ester solvent may be selected from the group comprising acetate, isopropyl acetate, and butyl acetate. The alcohol solvent may be selected from the group comprising ethanol, methanol, n-propanol, and butanol. Preferably, the solvent is acetone. The trifluoroacetic acid may be used as a solid or in solution form with acetone.

The salt of dabigatran etexilate is prepared at a temperature of about 10° C. to about 40° C., for example, about 15° C. to about 25° C. The salt of dabigatran etexilate is prepared in about 3 hours to about 6 hours, for example, about 4 hours to about 6 hours.

The dabigatran etexilate of Formula II or its salt, for example, trifluoroacetic acid salt, may be isolated by filtration, decantation, evaporation, distillation, or combinations thereof.

A third aspect of the present invention provides a process for the preparation of the methanesulfonate salt of dabigatran etexilate of Formula I

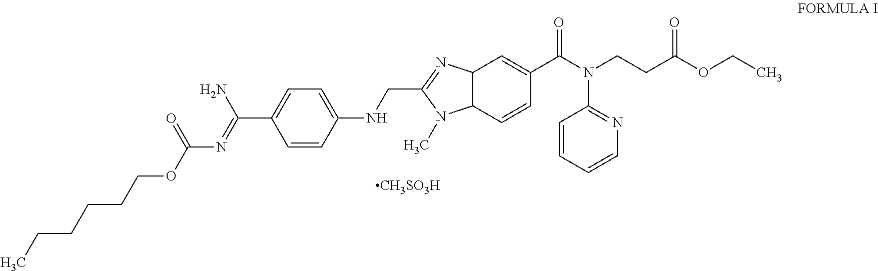

or salt thereof wherein the process comprises:
a) reacting the compound of Formula VI

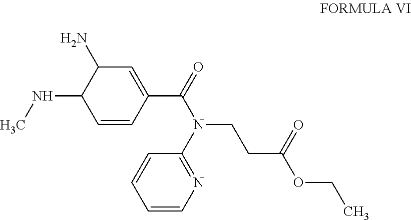 [SMILES: CCOC(=O)CCN(C(=O)C1=CC(N)C(NC)C=C1)c1ccccn1]

with the compound of Formula VII

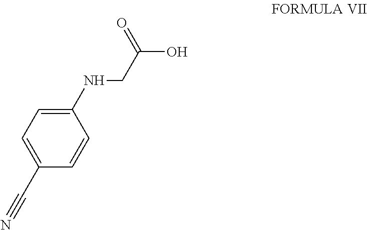 [SMILES: N#Cc1ccc(NCC(=O)O)cc1]

to prepare the compound of Formula VIII;

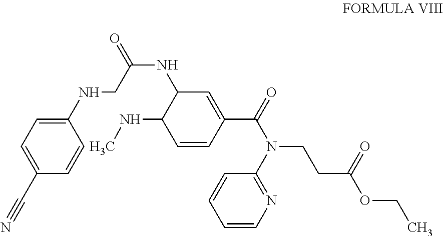 [SMILES: CCOC(=O)CCN(C(=O)C1=CC(NC(=O)CNc2ccc(C#N)cc2)C(NC)C=C1)c1ccccn1]

b) optionally isolating the compound of Formula VIII from the reaction mixture;
c) converting the compound of Formula VIII to the compound of Formula IX;

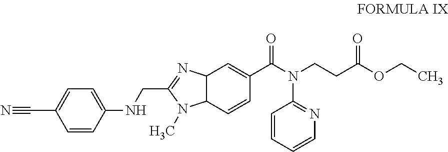 [SMILES: CCOC(=O)CCN(C(=O)C1=CC2N=C(CNc3ccc(C#N)cc3)N(C)C2C=C1)c1ccccn1]

d) converting the compound of Formula IX to the acetic acid salt of dabigatran ethyl ester of Formula V;
e) optionally isolating the acetic acid salt of dabigatran ethyl ester of Formula V from the reaction mixture;
f) reacting the acetic acid salt of dabigatran ethyl ester of Formula V with a reagent capable of providing etexilate group;
g) optionally isolating the dabigatran etexilate of Formula II or salt thereof from the reaction mixture;
h) converting dabigatran etexilate of Formula II or salt thereof to methanesulfonate salt of dabigatran etexilate of Formula I; and
i) isolating the methanesulfonate salt of dabigatran etexilate of Formula I from the reaction mixture.

The compound of Formula VI and the compound of Formula VII may be reacted as described in the first aspect. The compound of Formula VIII obtained by the reaction of the compound of Formula VI and the compound of Formula VII may optionally be isolated. The compound of Formula VIII may be converted to the compound of Formula IX as described in the first aspect. The compound of Formula IX may optionally be isolated by filtration, decantation, evaporation, distillation, or combinations thereof.

The compound of Formula IX may optionally be converted to the compound of Formula X as described in the first aspect. The compound of Formula IX or the compound of Formula X may be converted to the acetic acid salt of dabigatran ethyl ester of Formula V as described in the first aspect. The acetic acid salt of dabigatran ethyl ester of Formula V may exist in the form of the mono-acetate or di-acetate. The acetic acid salt of dabigatran ethyl ester of Formula V may be isolated by filtration, decantation, evaporation, distillation, or combinations thereof.

The acetic acid salt of dabigatran ethyl ester of Formula V may be converted to dabigatran etexilate of Formula II or its salt, for example, trifluoroacetic acid salt, as described in the second aspect.

The dabigatran etexilate of Formula II or its salt, for example, trifluoroacetic acid salt, may be isolated by filtration, decantation, evaporation, distillation, or combinations thereof.

The dabigatran etexilate of Formula II or its salt, for example, trifluoroacetic acid salt, is converted to the methanesulfonate salt of dabigatran etexilate. The dabigatran etexilate of Formula II or its salt, for example, trifluoroacetic acid salt, is treated with a solvent and a base before treating with methanesulfonic acid. The solvent may be selected from the group consisting of halogenated hydrocarbons, esters, ketones, alcohols, or mixtures thereof. The halogenated hydrocarbon may be dichloromethane. The ester solvent may be selected from the group comprising ethyl acetate, isopropyl acetate, and butyl acetate. The ketone solvent may be selected from the group comprising acetone, methyl butyl ketone, and methyl isopropyl ketone. The alcohol may be selected from the group comprising ethanol, methanol, n-propanol, and butanol. Preferably, the solvent is dichloromethane, ethyl acetate, or mixtures thereof.

The base may be an inorganic base or an organic base. The inorganic base may be selected from the group comprising sodium carbonate and potassium carbonate. The organic base may be selected from the group comprising ethyl amine, isopropyl amine, and diisopropylethyl amine. Preferably, the base is sodium carbonate or potassium carbonate. The dabigatran etexilate of Formula II or its salt, preferably trifluoroacetate salt, is treated with a solvent and a base at a temperature of about 10° C. to about 80° C., for example, about 20° C. to about 60° C. The dabigatran etexilate of Formula II or its salt, preferably trifluoroacetate salt, is treated with a solvent and a base for about 30 minutes to about 3 hours, for example, about 1 hour to about 2 hours.

The dabigatran etexilate of Formula II or its salt, for example, trifluoroacetic acid salt is treated with methanesulfonic acid in the presence of a solvent selected from the group consisting of ketones, esters, alcohols, or mixtures thereof. The ketone solvent may be selected from the group comprising acetone, methyl butyl ketone, and methyl isopropyl ketone. The ester solvent may be selected from the group comprising ethyl acetate, isopropyl acetate, and butyl acetate. The alcohol solvent may be selected from the group comprising ethanol, methanol, n-propanol, and butanol. Preferably, the solvent is ethyl acetate. The methanesulfonic acid may be used as a solid or in solution form with ethyl acetate.

The dabigatran etexilate of Formula II or its salt, for example, the trifluoroacetic acid salt, is treated with methanesulfonic acid at a temperature of about 10° C. to about 60° C., for example, about 20° C. to about 50° C. The reaction mixture obtained in step a) is treated with methanesulfonic acid for about 3 hours to about 6 hours, for example, about 4 hours to about 6 hours.

The methanesulfonate salt of dabigatran etexilate may be isolated by filtration, decantation, evaporation, distillation, or a combination thereof. The methanesulfonate salt of dabigatran etexilate may be characterized by XRPD pattern.

A fourth aspect of the present invention provides a process for the preparation of the acetic acid salt of dabigatran ethyl ester of Formula V

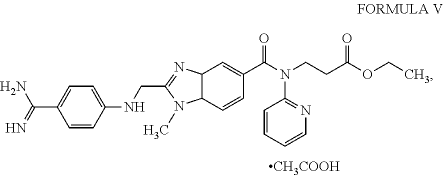

wherein the process comprises:
a) converting the compound of Formula IX

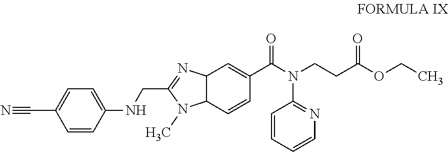 [SMILES: CCOC(=O)CCN(C(=O)C1=CC2N=C(CNc3ccc(C#N)cc3)N(C)C2C=C1)c1ccccn1]

to the compound of Formula X;

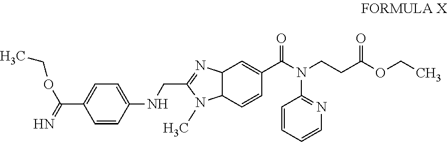 [SMILES: CCOC(=N)c1ccc(NCC2=NC3C=C(C(=O)N(CCC(=O)OCC)c4ccccn4)C=CC3N2C)cc1]

b) converting the compound of Formula X to the acetic acid salt of dabigatran ethyl ester of Formula V; and
c) isolating the acetic acid salt of dabigatran ethyl ester of Formula V from the reaction mixture.

The compound of Formula IX may be prepared as described in the first aspect. The compound of Formula IX may optionally be converted to the compound of Formula X as described in the first aspect.

The compound of Formula X may be converted to the acetic acid salt of dabigatran ethyl ester of Formula V as described in the first aspect. The acetic acid salt of dabigatran ethyl ester of Formula V may exist in the form of mono-acetate or di-acetate. The acetic acid salt of dabigatran ethyl ester of Formula V may be isolated by filtration, decantation, evaporation, distillation, or combinations thereof.

A fifth aspect of present invention provides the use of the acetic acid salt of dabigatran ethyl ester of Formula V for the preparation of dabigatran etexilate or salt thereof.

A sixth aspect of present invention provides the use of the trifluoroacetic acid salt of dabigatran etexilate of Formula XI for the preparation of methanesulfonate salt of dabigatran etexilate.

A seventh aspect of the present invention provides the trifluoroacetate salt of dabigatran etexilate of Formula XI.

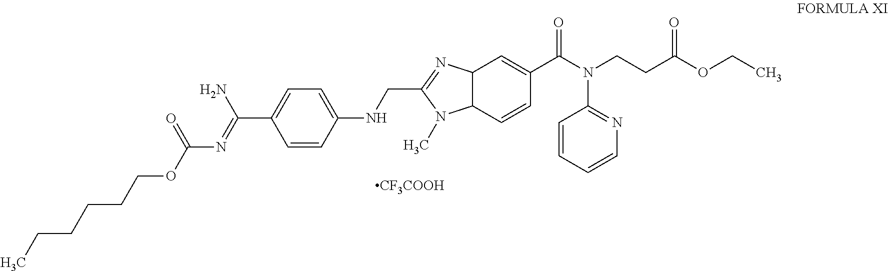 [SMILES: CCCCCCOC(=O)/N=C(\N)c1ccc(NCC2=NC3C=C(C(=O)N(CCC(=O)OCC)c4ccccn4)C=CC3N2C)cc1 ; "·CF3COOH"]

An eighth aspect of the present invention provides a process for the preparation of the trifluoroacetate salt of dabigatran etexilate of Formula XI, wherein the process comprises:
a) contacting ethyl N-[(2-{[(4-carbamimidoylphenyl)amino]methyl}-1-methyl-3a,7a-dihydro-1H-benzimidazol-5-yl)carbonyl]-N-pyridin-2-yl-β-alaninate of Formula III

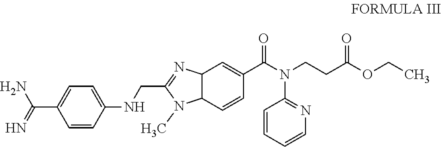 [SMILES: CCOC(=O)CCN(C(=O)C1=CC2N=C(CNc3ccc(C(=N)N)cc3)N(C)C2C=C1)c1ccccn1]

or its salt with n-hexyl chloroformate;
b) treating the reaction mixture obtained in step a) with trifluoroacetic acid; and
c) isolating the trifluoroacetate salt of dabigatran etexilate of Formula XI from the mixture thereof.

The ethyl N-[(2-{[(4-carbamimidoylphenyl)amino]methyl}-1-methyl-3a,7a-dihydro-1H-benzimidazol-5-yl)carbonyl]-N-pyridin-2-yl-β-alaninate of Formula III or its salt may be prepared according to the method provided in literature, for example, U.S. Pat. No. 6,087,380.

The salts of the compound of ethyl N-[(2-{[(4-carbamimidoylphenyl)amino]methyl}-1-methyl-3a,7a-dihydro-1H-benzimidazol-5-yl)carbonyl]-N-pyridin-2-yl-β-alaninate of Formula III may be selected from the hydrochloride, hydrobromide, or acetate salts. Preferably, the salt of compound of Formula III is an acetate salt.

The compound of Formula III or its salt is contacted with the n-hexyl chloroformate in the presence of a solvent selected from the group consisting of water, ethers, halogenated hydrocarbons, esters, or mixtures thereof. The ether solvent may be selected from the group comprising tetrahydrofuran, diisopropyl ether, and methyl t-butyl ether. The halogenated hydrocarbon solvent may be dichloromethane. The ester solvent may be ethyl acetate. Preferably, the solvent is tetrahydrofuran, either alone or in combination with water. The n-hexyl chloroformate may be used either as a solid or in solution form with tetrahydrofuran.

The compound of Formula III or its salt is contacted with n-hexyl chloroformate in the presence of an organic base or an inorganic base. The organic base may be selected from the group comprising ethylamine and diisopropyl ethyl amine. The inorganic base may be selected from the group comprising sodium carbonate and potassium carbonate. Preferably, the base is potassium carbonate.

The compound of Formula III or its salt is contacted with n-hexyl chloroformate at a temperature of about 10° C. to about 40° C., for example, about 15° C. to about 25° C. The compound of Formula V is contacted with n-hexyl chloroformate for about 3 hours to about 6 hours, for example, about 4 hours to about 6 hours.

The reaction mixture may be subjected to carbon treatment. The reaction mixture may optionally be treated with butylated hydroxytoluene. The solvent may be recovered from the reaction mixture and the reaction mixture used as such for the next step.

The reaction mixture obtained in step a) is treated with trifluoroacetic acid in the presence of a solvent selected from the group consisting of ketones, esters, alcohols, or mixtures thereof. The ketone solvent may be selected from the group comprising acetone, methyl butyl ketone, and methyl isopropyl ketone. The ester solvent may be selected from the group comprising ethyl acetate, isopropyl acetate, and butyl acetate. The alcohol solvent may be selected from the group comprising ethanol, methanol, n-propanol, and butanol. Preferably, the solvent is acetone. The trifluoroacetic acid may be used as a solid or in solution form with acetone.

The reaction mixture obtained in step a) is treated with trifluoroacetic acid at a temperature of about 10° C. to about 40° C., for example, about 15° C. to about 25° C. The reaction mixture obtained in step a) is treated with the trifluoroacetic acid for about 3 hours to about 6 hours, for example, about 4 hours to about 6 hours.

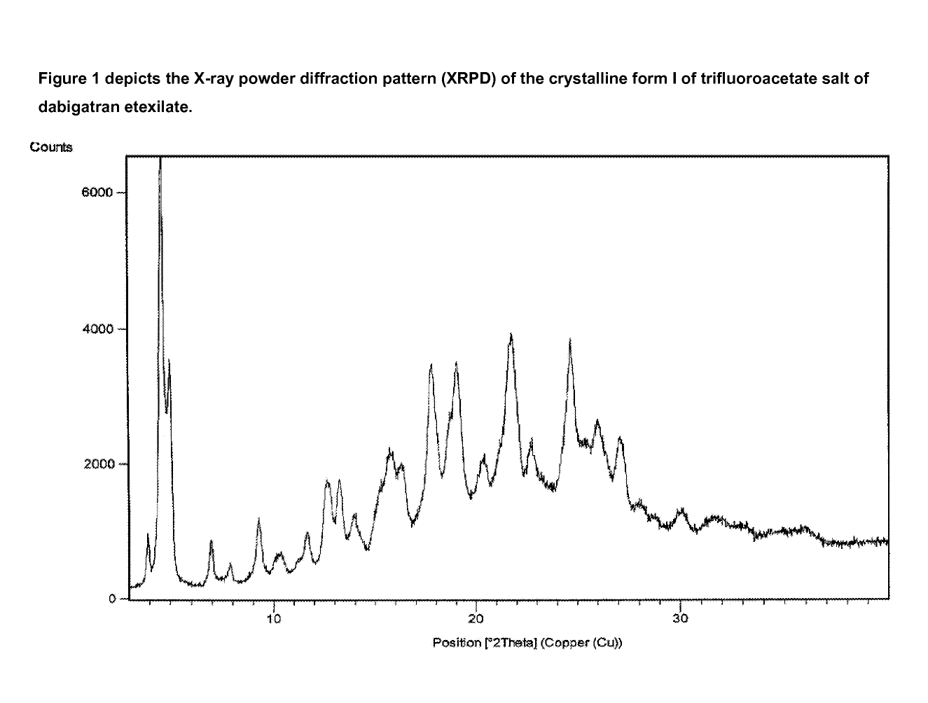

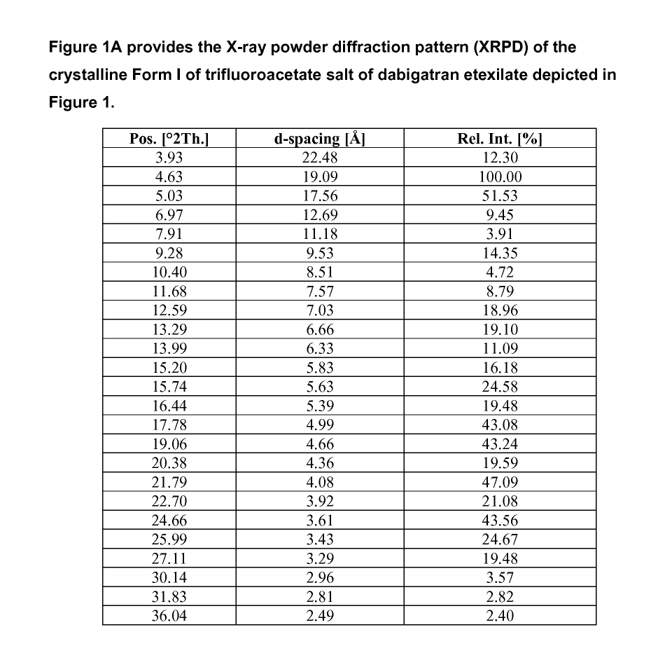

The trifluoroacetate salt of dabigatran etexilate may be isolated by filtration, decantation, evaporation, distillation, or combinations thereof. The trifluoroacetate salt of dabigatran etexilate has substantially the same XRPD pattern as depicted in FIG. 1, and is referred to herein as crystalline Form I of trifluoroacetate salt of dabigatran etexilate.

A ninth aspect of the present invention provides crystalline Form I of trifluoroacetate salt of dabigatran etexilate.

The crystalline Form I of trifluoroacetate salt of dabigatran etexilate has substantially the same XRPD (X-ray powder diffraction) pattern as depicted in FIG. 1.

The crystalline Form I of trifluoroacetate salt of dabigatran etexilate salt of Formula XI is characterized by an XRPD pattern having interplanar spacing (d) values substantially at 19.09, 17.56, 4.99, 4.66, 4.08, and 3.61 Å. The crystalline Form I of the trifluoroacetate salt of dabigatran etexilate salt of Formula XI is further characterized by an XRPD pattern having interplanar spacing (d) values substantially at 22.48, 19.09, 17.56, 12.69, 11.18, 9.53, 8.51, 7.57, 7.03, 6.66, 6.33, 5.83, 5.63, 5.39, 4.99, 4.66, 4.36, 4.08, 3.92, 3.61, 3.43, 3.29, 2.96, 2.81, and 2.49 Å.

A tenth aspect of the present invention provides a process for the purification of the trifluoroacetate salt of dabigatran etexilate, wherein the process comprises:
a) treating the trifluoroacetate salt of dabigatran etexilate of Formula XI with an alcohol solvent; and
b) isolating purified trifluoroacetate salt of dabigatran etexilate of Formula XI from the mixture thereof.

The alcohol solvent used for the purification may be selected from the group comprising methanol, ethanol, isopropanol, n-propanol, or mixtures thereof. Preferably, the alcohol solvent is ethanol. The trifluoroacetate salt of dabigatran etexilate may be treated with alcohol solvent at a temperature of about 10° C. to about 70° C., for example, about 20° C. to about 60° C. The trifluoroacetate salt of dabigatran etexilate may be treated with an alcohol solvent for about 2 hours to about 6 hours, for example, about 3 hours to about 4 hours.

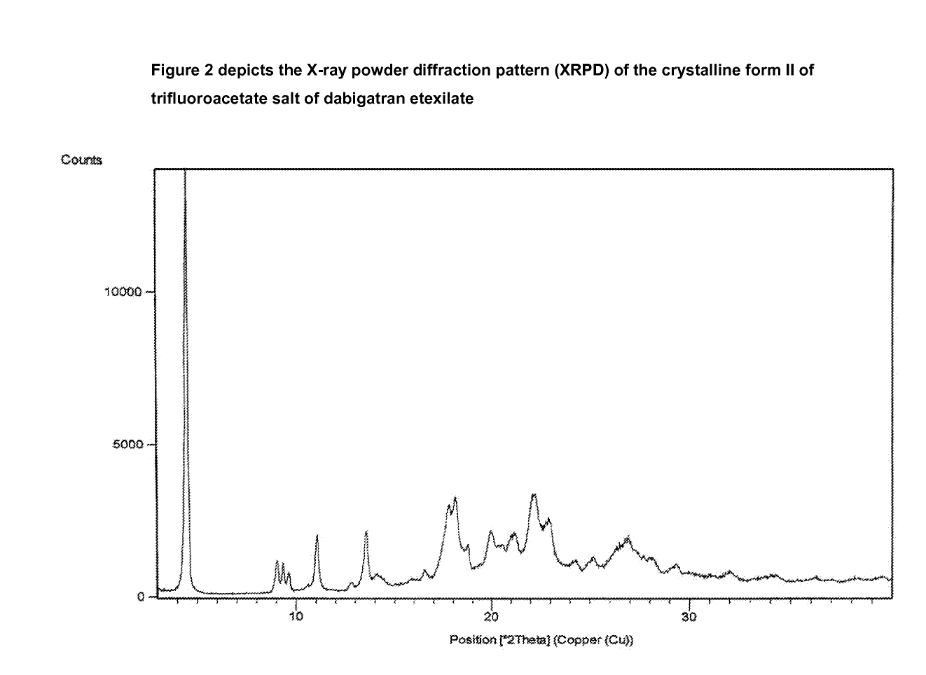

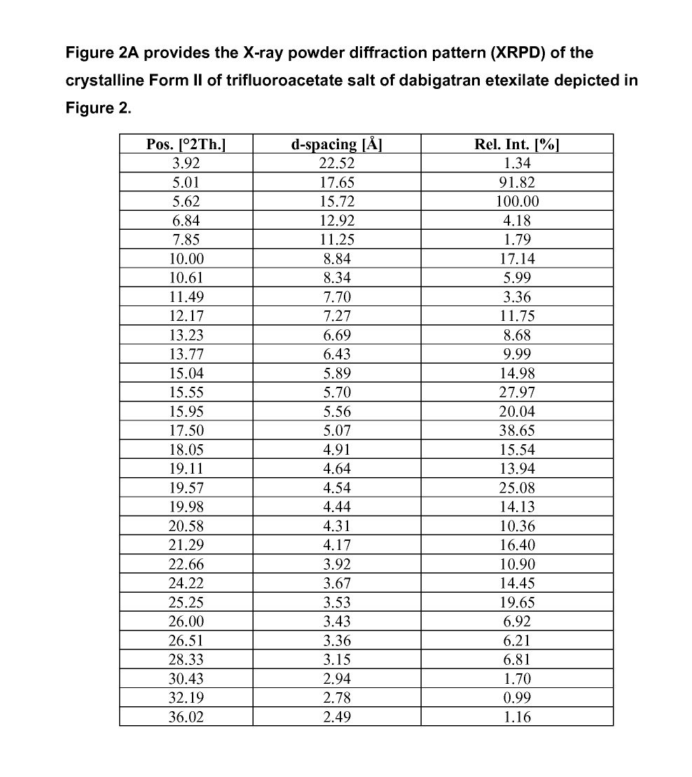

The purified trifluoroacetate salt of dabigatran etexilate may be isolated by filtration, decantation, evaporation, distillation, or combinations thereof. The purified trifluoroacetate salt of dabigatran etexilate has substantially the same XRPD pattern as depicted in FIG. 2 and is referred to herein as crystalline Form II of the trifluoroacetate salt of dabigatran etexilate salt of Formula XI.

An eleventh aspect of the present invention provides crystalline Form II of the trifluoroacetate salt of dabigatran etexilate salt of Formula XI.

The crystalline Form II of the trifluoroacetate salt of dabigatran etexilate salt of Formula XI has substantially the same XRPD (X-ray powder diffraction) pattern as depicted in FIG. 2. The crystalline Form II of trifluoroacetate salt of dabigatran etexilate salt of Formula XI is characterized by an XRPD pattern having interplanar spacing (d) values substantially at 17.65, 15.72, 5.70, 5.07, and 4.54 Å. The crystalline Form II of the trifluoroacetate salt of dabigatran etexilate salt of Formula XI is further characterized by an XRPD pattern having interplanar spacing (d) values substantially at 22.52, 17.65, 15.72, 12.92, 11.25, 8.84, 8.34, 7.70, 7.27, 6.69, 6.43, 5.89, 5.70, 5.56, 5.07, 4.91, 4.64, 4.54, 4.44, 4.31, 4.17, 3.92, 3.67, 3.53, 3.43, 3.36, 3.15, 2.94, 2.78, and 2.49 Å.

A twelfth aspect of the present invention provides a process for the preparation of the methanesulfonate salt of dabigatran etexilate, wherein the process comprises:
a) treating the trifluoroacetate salt of dabigatran etexilate of Formula XI with methanesulfonic acid; and
b) isolating methanesulfonate salt of dabigatran etexilate from the mixture thereof.

The trifluoroacetate salt of dabigatran etexilate of Formula XI may be treated with a suitable acid to prepare the pharmaceutically acceptable salts of dabigatran etexilate. Pharmaceutically acceptable salts of dabigatran etexilate may be, for example, methanesulfonate salt of dabigatran etexilate. The trifluoroacetate salt of dabigatran etexilate of Formula XI is treated with a solvent and a base before treating with the methanesulfonic acid. The solvent is selected from the group consisting of halogenated hydrocarbons, esters, ketones, alcohols, or mixtures thereof. The halogenated hydrocarbon may be, for example, dichloromethane. The ester solvent may be selected from the group comprising ethyl acetate, isopropyl acetate, and butyl acetate. The ketone solvent may be selected from the group comprising acetone, methyl butyl ketone, and methyl isopropyl ketone. The alcohol solvent may be selected from the group comprising ethanol, methanol, n-propanol, and butanol. Preferably, the solvent is dichloromethane, ethyl acetate, or a mixture thereof.

The base may be an inorganic base or an organic base. The inorganic base may be selected from the group comprising sodium carbonate and potassium carbonate. The organic base may be selected from the group comprising ethyl amine, isopropyl amine, and diisopropylethyl amine. Preferably, the base is sodium carbonate or potassium carbonate. The trifluoroacetate salt of dabigatran etexilate of Formula XI is treated with a solvent and a base at a temperature of about 10° C. to about 80° C., for example, about 20° C. to about 60° C. The trifluoroacetate salt of dabigatran etexilate of Formula XI is treated with a solvent and a base for about 30 minutes to about 3 hours, for example, about 1 hour to about 2 hours.

The trifluoroacetate salt of dabigatran etexilate of Formula XI is treated with methanesulfonic acid in the presence of a solvent selected from the group consisting of ketones, esters, alcohols, or mixtures thereof. The ketone solvent may be selected from the group comprising acetone, methyl butyl ketone, and methyl isopropyl ketone. The ester solvent may be selected from the group comprising ethyl acetate, isopropyl acetate, and butyl acetate. The alcohol solvent may be selected from the group comprising ethanol, methanol, n-propanol, and butanol. Preferably, the solvent is ethyl acetate. The methanesulfonic acid may be used as a solid or in solution form with ethyl acetate.

The trifluoroacetate salt of dabigatran etexilate is treated with methanesulfonic acid at a temperature of about 10° C. to about 60° C., for example, about 20° C. to about 50° C. The trifluoroacetate salt of dabigatran etexilate is treated with methanesulfonic acid for about 3 hours to about 6 hours, for example, about 4 hours to about 6 hours.

The methanesulfonate salt of dabigatran etexilate may be isolated by filtration, decantation, evaporation, distillation, or combinations thereof.

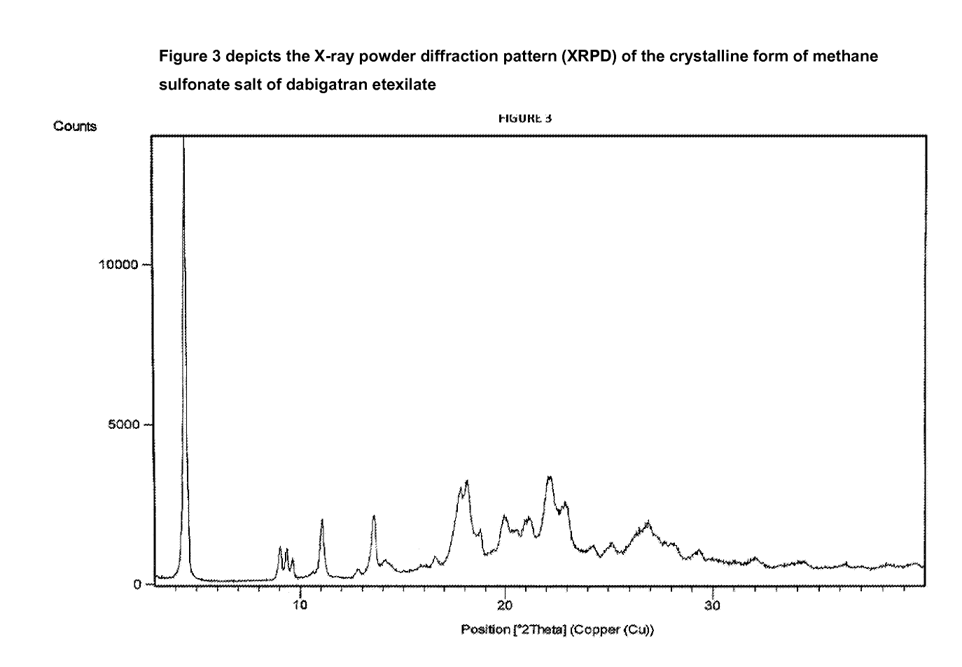

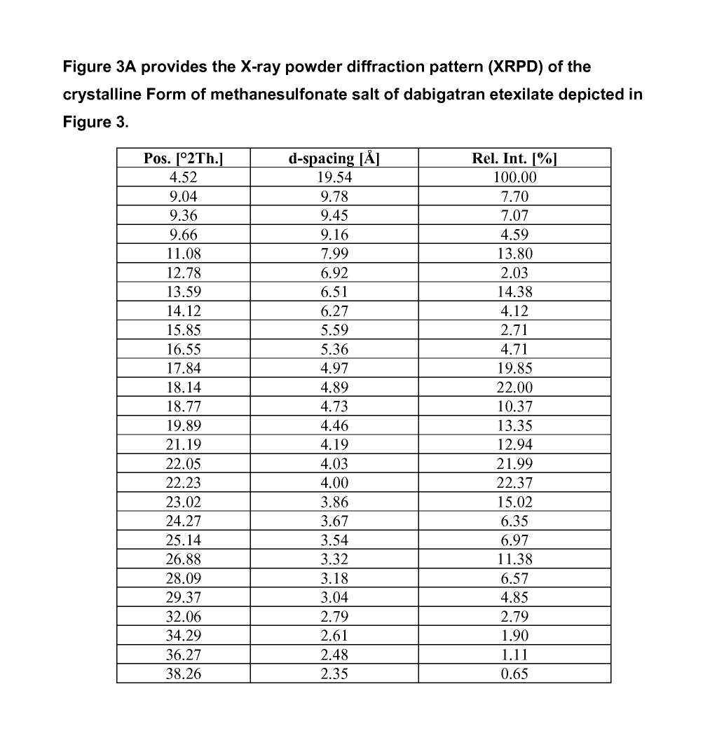

The methanesulfonate salt of dabigatran etexilate prepared by the present invention has substantially the same XRPD (X-ray powder diffraction) pattern as depicted in FIG. 3. The methanesulfonate salt of dabigatran etexilate is characterized by an XRPD pattern having interplanar spacing (d) values substantially at 19.54, 4.97, 4.89, 4.03, and 4.00 Å. The methanesulfonate salt of dabigatran etexilate is further characterized by an XRPD pattern having interplanar spacing (d) values substantially at 19.54, 9.78, 9.45, 9.16, 7.99, 6.92, 6.51, 6.27, 5.59, 5.36, 4.97, 4.89, 4.73, 4.46, 4.19, 4.03, 4.00, 3.86, 3.67, 3.54, 3.32, 3.18, 3.04, 2.79, 2.61, 2.48, and 2.35 Å.

The XRPD of the samples were determined by using a PANalytical X'Pert PRO X-Ray Powder Diffractometer in the range 3-40 degree 2 theta and under tube voltage and current of 45 Kv and 40 mA, respectively. Copper radiation of wavelength 1.54 angstrom and X'Celerator detector was used.

While the present invention has been described in terms of its specific embodiments, certain modifications and equivalents will be apparent to those skilled in the art and are intended to be included within the scope of the present invention.

### EXAMPLES

### Example 1

### Preparation of ethyl n-[(2-{[(4-cyanophenyl)amino]methyl}-1-methyl-3a,7a-dihydro-1H-benzimidazol-5-yl)carbonyl]-N-pyridin-2-yl-β-alaninate (compound of Formula IX)

N,N-Carbonyl diimidazole (5.82 g) and 2-[(4-Cyanophenyl)amino]acetic acid (compound of Formula VII) (6.28 g) were added to tetrahydrofuran (120 mL) at 20° C. to 25° C. Ethyl N-{[3-amino-4-(methylamino)cyclohexa-1,5-dien-1-yl]carbonyl}-N-pyridin-2-yl-β-alaninate (compound of Formula VI) (10 g) was added to the reaction mixture and the temperature of the reaction mixture was increased to 50° C. to 55° C. Butylated hydroxyl toluene (1 g) was added to the reaction mixture and tetrahydrofuran was recovered under vacuum at 55° C.

Acetic acid (40 mL) was added to the reaction mixture and stirred at 90° C. to 95° C. until the reaction was complete. Acetic acid was completely recovered under vacuum at 65° C. to 70° C. The reaction mixture was cooled to 20° C. to 25° C. De-ionized water (50 mL) was added and the aqueous layer was extracted with ethyl acetate (2×50 mL). The organic layer was washed with 5% aqueous sodium bicarbonate solution (70 mL). Ethyl acetate was recovered under vacuum at 55° C. to 60° C. 2-Propanol (60 mL) was added to the reaction mixture and stirred at 55° C. to 60° C. The reaction mixture was cooled to 20° C. to 25° C. and stirred for 16 hours at 20° C. to 25° C. The reaction mixture was filtered and washed with 2-propanol (10 mL). The wet material was dried under vacuum to obtain the title compound.

Yield: 8.35 g

### Example 2

### Preparation of the Acetate Salt of Dabigatran Ethyl Ester (Compound of Formula V)

Ethyl N-[(2-{[(4-cyanophenyl)amino]methyl}-1-methyl-3a,7a-dihydro-1H-benzimidazol-5-yl)carbonyl]-N-pyridin-2-yl-β-alaninate (compound of Formula IX) (100 g) was added to dimethylformamide (250 mL). Hydrochloric acid gas dissolved in ethanol (250 mL) was added to the reaction mixture for 10 hours at 0° C. to −10° C. The reaction mixture was stirred until the reaction was complete. Aqueous potassium carbonate solution was added to the reaction mixture and extracted using dichloromethane (1500 mL) and washed with 25% sodium chloride solution (500 mL).

Dichloromethane was recovered completely under vacuum. Ethanol (700 mL) was added to the reaction mixture followed by the addition of ammonium acetate (104 g) and the mixture was stirred until the reaction was complete. Ethanol was recovered under vacuum. Acetone (300 mL) was added to the reaction mixture and recovered completely under vacuum. Deionized water (1000 mL) was added to the reaction mixture and stirred for 2 hours at 25° C. The reaction mixture was cooled to 5° C. to 10° C. stirred, filtered and dried under vacuum at 45° C. to 50° C. to obtain the title compound.

Yield: 85 g

### Example 3

### Preparation of Dabigatran Etexilate Trifluoroacetate Salt

Acetate salt of dabigatran ethyl ester (50 g) was added to tetrahydrofuran (750 mL) and deionized water (250 mL). Potassium carbonate (37.08 g) was added to the reaction mixture and stirred for 30 minutes. A solution of n-hexyl chloroformate (16.19 g) dissolved in tetrahydrofuran (250 mL) was added to the reaction mixture at 18° C. to 20° C. and stirred for 2 hours at 20° C. to 22° C. The layers were separated and butylated hydroxytoluene (BHT) (0.5 g) was added to the tetrahydrofuran layer. Tetrahydrofuran was recovered under vacuum.

Acetone (150 mL) was added to the reaction mixture and stirred for 20 minutes. The acetone was recovered under vacuum. The solid obtained was dissolved in acetone (392 mL). A solution of trifluoroacetic acid (9.66 g) in acetone (56 mL) was added to the reaction mixture at 18° C. to 20° C. The reaction mixture was stirred at 20° C. to 22° C. for 2 hours, filtered, and dried at 55° C. to obtain the title compound having XRPD data as depicted in FIG. 1.

Yield: 53 g

### Example 4

### Purification of Dabigatran Etexilate Trifluoroacetate Salt

Dabigatran etexilate trifluoroacetate salt (50 g) obtained in Example 3 was dissolved in ethanol (350 mL) at 55° C. for 15 minutes to 20 minutes. The reaction mixture was cooled to 10° C. to 15° C. for 20 minutes to 25 minutes. The reaction mixture was stirred for 2 hours at 20° C., filtered, and dried under vacuum. The reaction mixture was washed with ethanol (50 mL), and then dried under vacuum at 55° C. for 15 hours to obtain the title compound having XRPD data as depicted in FIG. 2.

Yield: 39 g

(M+H)⁺: m/z=628.5

¹H NMR (400 MHz, CDCl₃): δ 0.86-0.89 (t, 3H), 1.10-1.13 (t, 3H), 1.28-1.30 (m, 6H), 1.67 (m, 2H), 2.66-2.69 (t, 2H), 3.77 (s, 3H), 3.94-3.98 (t, 2H), 4.22-4.25 (m, 4H), 4.68-4.69 (d, 2H), 6.85-6.89 (m, 3H), 6.90-7.17 (m, 2H), 7.40-7.42 (m, 2H), 7.47 (t, 1H), 7.64-7.67 (dt, 3H), 8.37 (dd, 1H), 10.0 (s, 1H), 10.65 (bs, 1H), 11.90 (bs, 1H).

### Example 5

### Preparation of Dabigatran Etexilate Methanesulfonate Salt

Dabigatran etexilate trifluoroacetate salt (100 g) was dissolved in dichloromethane (2000 mL) at 25° C. The reaction mixture was washed with aqueous potassium carbonate solution (200 g in 200 mL de-ionized water). Dichloromethane was recovered from the reaction mixture under vacuum. Ethyl acetate (1700 mL) was added to the reaction mixture and stirred for 10 minutes. The reaction mixture was heated to 65° C. to 70° C. and further cooled to 40° C. Methanesulphonic acid solution (11.01 g methanesulphonic acid dissolved in 200 mL ethyl acetate) was added to the reaction mixture drop-wise at 38° C. to 40° C. The reaction mixture was stirred at 20° C. to 25° C. for 2 hours, filtered, and dried under vacuum at 45° C. to 50° C. to obtain the title compound.

Yield: 80 g

### Example 6

### Preparation of Dabigatran Etexilate Methanesulfonate Salt

Dabigatran etexilate trifluoroacetate salt (35 g) was dissolved in dichloromethane (350 mL) at 25° C. A 5% aqueous sodium carbonate solution (210 mL) was added to the reaction mixture and stirred for 10 minutes. The dichloromethane layer was separated and dichloromethane was recovered under vacuum. Ethyl acetate (550 mL) was added to the reaction mixture and stirred for 10 minutes. Methanesulphonic acid solution (3.99 g methanesulphonic acid dissolved in 55 mL ethyl acetate) was added to the reaction mixture drop-wise at 20° C. to 25° C. The reaction mixture was stirred at 20° C. to 25° C. for 2 hours. The reaction mixture was filtered under vacuum and washed with ethyl acetate (27 mL). The solid obtained was dried under vacuum at 55° C. for 14 hours to 15 hours to obtain the title compound having XRPD data as depicted in FIG. 3.

Yield: 29.75 g.

1. A process for the preparation of acetic acid salt of dabigatran ethyl ester of Formula V 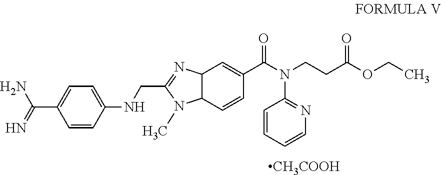
wherein the process comprises:
a) reacting the compound of Formula VI 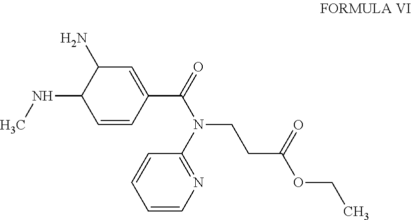 [SMILES: CCOC(=O)CCN(C(=O)C1=CC(N)C(NC)C=C1)c1ccccn1]
with the compound of Formula VII 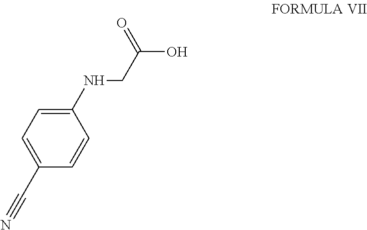 [SMILES: N#Cc1ccc(NCC(=O)O)cc1]
to prepare the compound of Formula VIII; 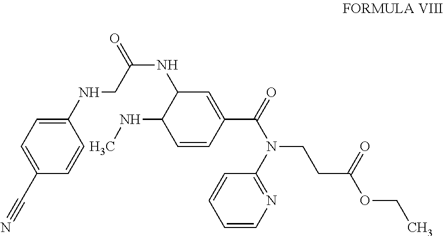 [SMILES: CCOC(=O)CCN(C(=O)C1=CC(NC(=O)CNc2ccc(C#N)cc2)C(NC)C=C1)c1ccccn1]
b) optionally isolating the compound of Formula VIII from the reaction mixture;
c) converting the compound of Formula VIII to the compound of Formula IX; 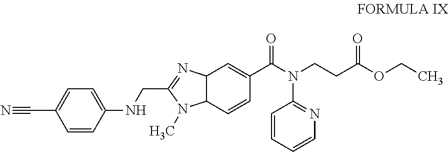 [SMILES: CCOC(=O)CCN(C(=O)C1=CC2N=C(CNc3ccc(C#N)cc3)N(C)C2C=C1)c1ccccn1]
d) converting the compound of Formula IX to the acetic acid salt of dabigatran ethyl ester of Formula V; and
e) isolating the acetic acid salt of dabigatran ethyl ester of Formula V from the reaction mixture.

2. A process for the preparation of dabigatran etexilate of Formula II 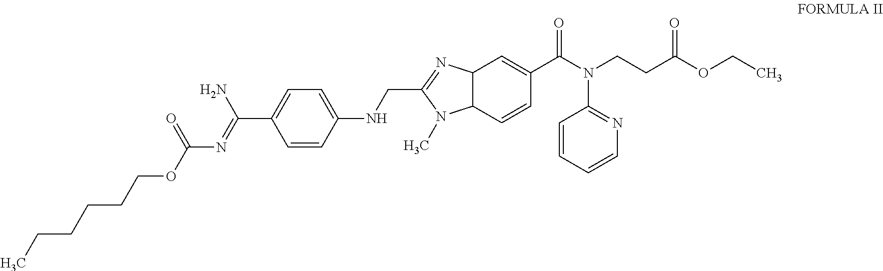 [SMILES: CCCCCCOC(=O)/N=C(\N)c1ccc(NCC2=NC3C=C(C(=O)N(CCC(=O)OCC)c4ccccn4)C=CC3N2C)cc1]
or a salt thereof wherein the process comprises:
a) reacting the compound of Formula VI 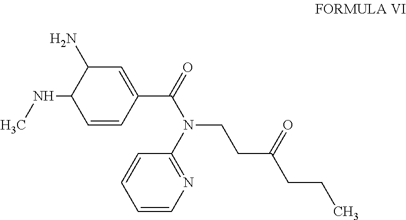 [SMILES: CCCC(=O)CCN(C(=O)C1=CC(N)C(NC)C=C1)c1ccccn1]
with the compound of Formula VII 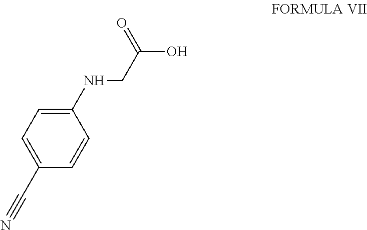 [SMILES: N#Cc1ccc(NCC(=O)O)cc1]
to prepare the compound of Formula VIII; 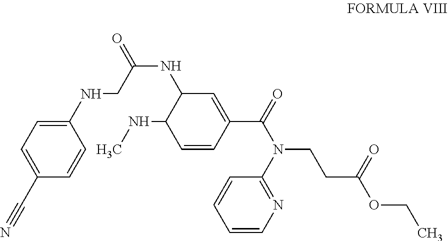 [SMILES: CCOC(=O)CCN(C(=O)C1=CC(NC(=O)CNc2ccc(C#N)cc2)C(NC)C=C1)c1ccccn1]
b) optionally isolating the compound of Formula VIII from the reaction mixture;
c) converting the compound of Formula VIII to the compound of Formula IX; 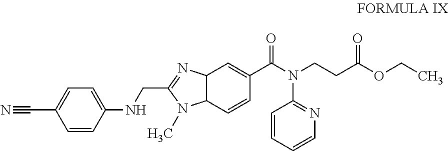 [SMILES: CCOC(=O)CCN(C(=O)C1=CC2N=C(CNc3ccc(C#N)cc3)N(C)C2C=C1)c1ccccn1]
d) converting the compound of Formula IX to the acetic acid salt of dabigatran ethyl ester of Formula V;
e) optionally isolating the acetic acid salt of dabigatran ethyl ester of Formula V from the reaction mixture;
f) reacting the acetic acid salt of dabigatran ethyl ester of Formula V with a reagent capable of providing etexilate group; and
g) isolating the dabigatran etexilate of Formula II or a salt thereof from the reaction mixture.

3. A process for the preparation of methanesulfonate salt of dabigatran etexilate of Formula I 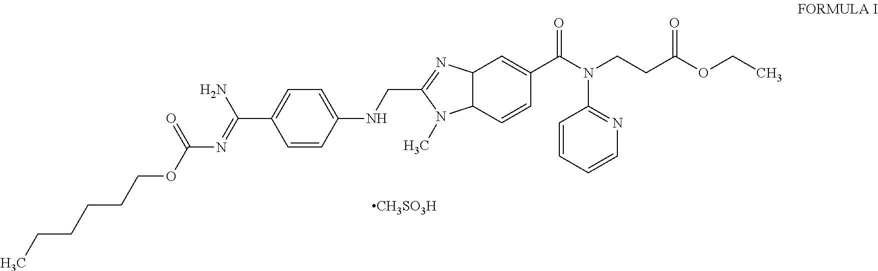 [SMILES: CCCCCCOC(=O)/N=C(\N)c1ccc(NCC2=NC3C=C(C(=O)N(CCC(=O)OCC)c4ccccn4)C=CC3N2C)cc1]
or a salt thereof wherein the process comprises:
a) reacting the compound of Formula VI 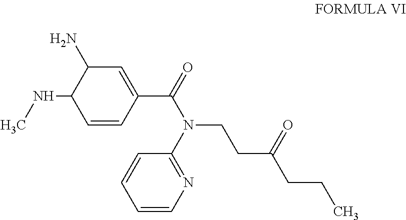 [SMILES: CCCC(=O)CCN(C(=O)C1=CC(N)C(NC)C=C1)c1ccccn1]
with the compound of Formula VII 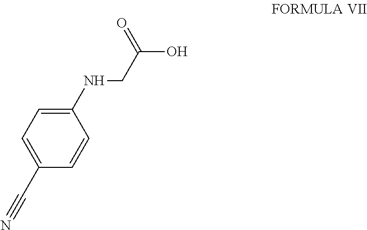 [SMILES: N#Cc1ccc(NCC(=O)O)cc1]
to prepare the compound of Formula VIII; 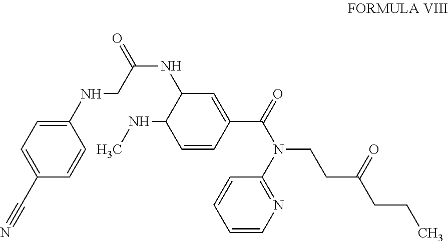 [SMILES: CCCC(=O)CCN(C(=O)C1=CC(NC(=O)CNc2ccc(C#N)cc2)C(NC)C=C1)c1ccccn1]
b) optionally isolating the compound of Formula VIII from the reaction mixture;
c) converting the compound of Formula VIII to compound of Formula IX; 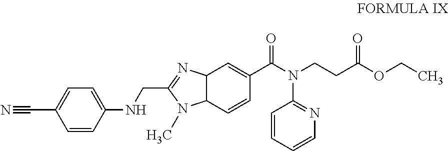 [SMILES: CCOC(=O)CCN(C(=O)C1=CC2N=C(CNc3ccc(C#N)cc3)N(C)C2C=C1)c1ccccn1]
d) converting the compound of Formula IX to the acetic acid salt of dabigatran ethyl ester of Formula V;
e) optionally isolating the acetic acid salt of dabigatran ethyl ester of Formula V from the reaction mixture;
f) reacting the acetic acid salt of dabigatran ethyl ester of Formula V with a reagent capable of providing etexilate group;
g) optionally isolating the dabigatran etexilate of Formula II or a salt thereof from the reaction mixture;
h) converting the dabigatran etexilate of Formula II or a salt thereof to methanesulfonate salt of dabigatran etexilate of Formula I; and
i) isolating the methanesulfonate salt of dabigatran etexilate of Formula I from the reaction mixture.

4. A process for the preparation of the acetic acid salt of dabigatran ethyl ester of Formula V 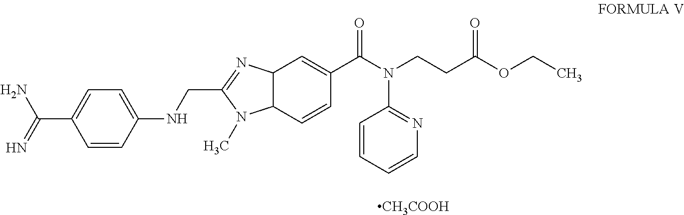
which comprises:
a) converting the compound of Formula IX 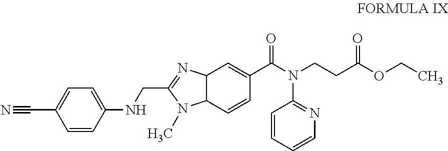 [SMILES: CCOC(=O)CCN(C(=O)C1=CC2N=C(CNc3ccc(C#N)cc3)N(C)C2C=C1)c1ccccn1]
to the compound of Formula X; 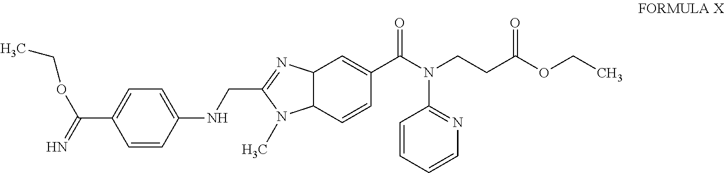 [SMILES: CCOC(=N)c1ccc(NCC2=NC3C=C(C(=O)N(CCC(=O)OCC)c4ccccn4)C=CC3N2C)cc1]
b) converting the compound of Formula X to the acetic acid salt of dabigatran ethyl ester of Formula V; and
c) isolating the acetic acid salt of dabigatran ethyl ester of Formula V from the reaction mixture.

5. The process according to claim 1, wherein the compound of Formula VI and the compound of Formula VII are reacted in the presence of a condensing agent and a solvent.

6-9. (canceled)

10. The process according to claim 1, wherein the compound of Formula VIII is converted to the compound of Formula IX in the presence of a cyclizing agent, and a solvent.

11-13. (canceled)

14. The process according to claim 1, wherein the compound of Formula IX is optionally converted to the compound of Formula X in the presence of hydrochloric acid and a solvent.

15-16. (canceled)

17. The process according to claim 1, wherein the compound of Formula IX is converted to acetic acid salt of dabigatran ethyl ester of Formula V in the presence of ammonium acetate and a solvent.

18-23. (canceled)

24. The process according to claim 3, wherein the dabigatran etexilate of Formula II or its salt is treated with a solvent and a base before treating with the methanesulfonic acid.

25. Use of the acetic acid salt of dabigatran ethyl ester of Formula V for the preparation of dabigatran etexilate or a salt thereof.

26. Use of the trifluoroacetic acid salt of dabigatran etexilate of Formula XI for the preparation of methanesulfonate salt of dabigatran etexilate.

27. A trifluoroacetate salt of dabigatran etexilate salt of Formula XI.

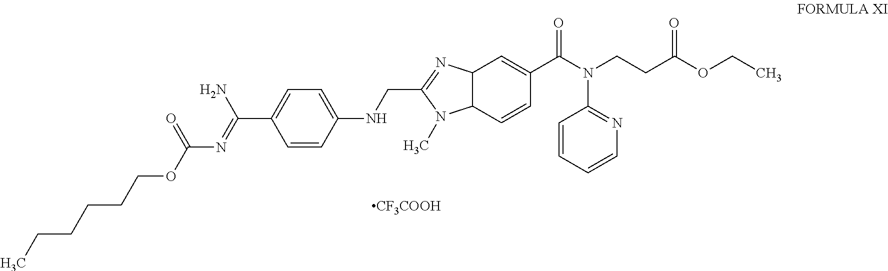 [SMILES: CCCCCCOC(=O)/N=C(\N)c1ccc(NCC2=NC3C=C(C(=O)N(CCC(=O)OCC)c4ccccn4)C=CC3N2C)cc1 ; "·CF3COOH"]

28. A process for the preparation of trifluoroacetate salt of dabigatran etexilate of Formula XI, wherein the process comprises:
a) contacting ethyl N-[(2-{[(4-carbamimidoylphenyl)amino]methyl}-1-methyl-3a,7a-dihydro-1H-benzimidazol-5-yl)carbonyl]-N-pyridin-2-yl-β-alaninate of Formula III 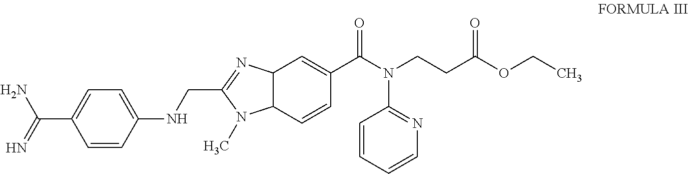 [SMILES: CCOC(=O)CCN(C(=O)C1=CC2N=C(CNc3ccc(C(=N)N)cc3)N(C)C2C=C1)c1ccccn1]
or its salt with n-hexyl chloroformate;
b) treating the reaction mixture obtained in step a) with trifluoroacetic acid; and
c) isolating the trifluoroacetate salt of dabigatran etexilate of Formula XI from the mixture thereof.

29-32. (canceled)

33. A process for the purification of the trifluoroacetate salt of dabigatran etexilate, wherein the process comprises:
a) treating the trifluoroacetate salt of dabigatran etexilate of Formula XI with an alcohol solvent; and
b) isolating purified trifluoroacetate salt of dabigatran etexilate of Formula XI from the mixture thereof.

34-37. (canceled)

38. A process for the preparation of methanesulfonate salt of dabigatran etexilate, wherein the process comprises:
a) treating trifluoroacetate salt of dabigatran etexilate of Formula XI with methanesulfonic acid; and
b) isolating methanesulfonate salt of dabigatran etexilate from the mixture thereof.

39. The process according to claim 2, wherein the compound of Formula VI and the compound of Formula VII are reacted in the presence of a condensing agent and a solvent.

40. The process according to claim 3, wherein the compound of Formula VI and the compound of Formula VII are reacted in the presence of a condensing agent and a solvent.

41. The process according to claim 2, wherein the compound of Formula VIII is converted to the compound of Formula IX in the presence of a cyclizing agent and a solvent.

42. The process according to claim 3, wherein the compound of Formula VIII is converted to the compound of Formula IX in the presence of a cyclizing agent and a solvent.

43. The process according to claim 2, wherein the compound of Formula IX is optionally converted to the compound of Formula X in the presence of hydrochloric acid and a solvent.

44. The process according to claim 3, wherein the compound of Formula IX is optionally converted to the compound of Formula X in the presence of hydrochloric acid and a solvent.

45. The process according to claim 4, wherein the compound of Formula IX is optionally converted to the compound of Formula X in the presence of hydrochloric acid and a solvent.

46. The process according to claim 2, wherein the compound of Formula IX is converted to acetic acid salt of dabigatran ethyl ester of Formula V in the presence of ammonium acetate and a solvent.

47. The process according to claim 3, wherein the compound of Formula IX is converted to acetic acid salt of dabigatran ethyl ester of Formula V in the presence of ammonium acetate and a solvent.

48. The process according to claim 4, wherein the compound of Formula X is converted to acetic acid salt of dabigatran ethyl ester of Formula V in the presence of ammonium acetate and a solvent.
